(19) United States
(12) Patent Application Publication
(10) Pub. No.: US 2026/0076886 A1
(43) Pub. Date: Mar. 19, 2026

(54) METHODS AND COMPOSITIONS FOR SKIN IMPROVEMENTS

(71) Applicant: NEWMEDICAL TECHNOLOGY, INC., Northbrook, IL (US)

(72) Inventors: Haitham Matloub, Northbrook, IL (US); Garrett P. Hoepker, Northbrook, IL (US)

(73) Assignee: NEWMEDICAL TECHNOLOGY, INC., Northbrook, IL (US)

(21) Appl. No.: 19/335,947

(22) Filed: Sep. 22, 2025

Related U.S. Application Data
(63) Continuation of application No. PCT/US2024/021197, filed on Mar. 22, 2024.
(60) Provisional application No. 63/491,741, filed on Mar. 23, 2023.

(51) Int. Cl. A61K 8/362; A61K 8/34; A61K 8/35; A61K 8/37; A61K 8/67; A61K 8/85; A61K 8/894; A61K 8/9789; A61Q 19/02

(52) U.S. Cl. CPC A61K 8/362; A61K 8/347; A61K 8/35; A61K 8/37; A61K 8/675; A61K 8/676; A61K 8/678; A61K 8/85; A61K 8/894; A61K 8/9789; A61Q 19/02; A61K 2800/5922; A61K 2800/805

(57) ABSTRACT

Described herein are compositions and formulations for the treatment of acne, hyperpigmentation, wrinkles, and combinations thereof. The compositions comprise active agents in amounts of from about 0.1-20% based on the total weight of the composition, and an active agent comprising at least one hyperpigmentation agent in an amount of 15 weight % or less. In some embodiments, the composition comprises acetyl zingerone, azelaic acid, bakuchiol, and licorice extract.

### CROSS REFERENCE TO RELATED APPLICATIONS

This application is a bypass continuation application of PCT International Application No. PCT/US2024/021197, filed Mar. 22, 2024, which claims priority to and the benefit of U.S. Patent Application No. 63/491,741, filed on Mar. 23, 2023, the contents of each of which are incorporated herein by reference in their entireties.

### FIELD OF DISCLOSURE

The present disclosure is directed to improved topical dermatological formulations and methods of using the same.

### BACKGROUND

A wide variety of topical formulations are used every day. In most cases, they do not provide sufficient delivery of compounds, agents, and/or formulations to address the comprehensive needs for skin improvements. Recent scientific advances that provide new insights into how sun damages skin make it apparent that even broad-spectrum sunscreens with high protection factors cannot fully protect skin from the types of damage which precede induction of cancer and photoaging as well as protect the microbiome to maintain skin in optimal health and condition throughout life.

Acne vulgaris is a ubiquitous condition affecting up to 80% of individuals aged 11 to 30 years, and is one of the most common diseases in the world. The chronic obstructive and inflammatory disease is caused by over secretion by the sebaceous glands. Acne and its sequelae can have a dramatic effect on quality of life, including contributing to higher social appearance anxiety. Additionally, the condition brings with it a significant likelihood of scarring, thought to be the result of a suboptimal wound healing response due to acne-associated inflammation.

Thus, there is a need for new compounds, agents, and/or formulations to address these needs.

### BRIEF SUMMARY

The skincare formula described herein is designed to tackle at least five different skin concerns at once: (i) skin lightening. (ii) dark spot reduction, (iii) skin retexturing, (iv) improving anti-inflammatory properties, and (v) protection against harmful microbiomes. The formula's antioxidant level plays a crucial role as it affects the stability of the ingredients both outside and inside the skin. The unique formulae of the present disclosure take into consideration ingredient interactions too. The carefully selected ingredients will not only be effective on their own but also work together synergistically without causing instability or major skin irritation. Texture of the formulations and molecular sizes of the ingredients are also important factors to consider. The texture of the product affects how it feels and applies to the skin, which can impact user satisfaction. The molecular size of the ingredients determines how well they can penetrate the skin to provide the intended benefits. In summary, the skincare formulae of the present disclosure are designed with a holistic approach, targeting multiple skin concerns with carefully selected ingredients that work in harmony. The focus on antioxidants, ingredient interactions, product texture, and molecular sizes of the ingredients ensure that the product is not only effective but also stable, safe, and pleasant to use.

In some embodiments, formulations described herein are designed for improving skin appearance by using a combination of ingredients to achieve necessary skin improvement targets including: (i) reducing dark spots and providing skin lightening, (ii) reducing skin inflammation, (iii) improving skin surface appearance/skin retexturing, (iv) protecting the skin's surface from oxidative damage caused by free radicals and environmental aggressors like UV and pollution of skin, and (v) controlling the harmful skin microbiome.

Also described herein are compositions comprising multiple ingredients that are combined to act together and complement the function of each other. Optimal skin improvement requires the use of multiple key ingredients that are capable of working together and safeguarding skin's integrity. Compositions described herein, when administered provide for: (i) skin lightening and reducing dark spots via tyrosinase inhibition; (ii) skin retexturing; (iii) anti-inflammatory action; (iv) antioxidant and skin protection; and (v) harmful microbiome control.

In some embodiments, skin improvement of a patient in need thereof is observed after or during administration of a composition described herein. In some embodiments, the present disclosure provides for methods of skin lightening. In some embodiments, the present disclosure provides for methods of reducing dark spots on the skin. In some embodiments, the present disclosure provides for methods of tyrosinase inhibition. In some embodiments, the present disclosure provides for methods of skin retexturing. In some embodiments, the present disclosure provides for methods of inducing an anti-inflammatory response on or in the skin of a patient in need thereof. In some embodiments, the present disclosure provides for methods of administering at least one of: a skin lightening agent, a dark spot-reducing agent, a tyrosinase inhibitor, an anti-inflammatory agent, a skin protectant, an agent involved in microbiome control, an antioxidant, or combinations of any of these. The present disclosure further provides for methods of reducing melanin levels in the skin of a subject, the method comprising administering the compositions described herein to the subject as a topical formulation. In some embodiments, the skin of the subject is lightened. In some embodiments, the skin of subject has a reduction of dark spots. In some embodiments, the skin of the subject is retextured. In some embodiments, the skin of the subject has improved anti-inflammatory properties. In some embodiments, the skin of the subject is protected against microbiomes.

The present disclosure provides for a composition including: about 0.1-2.0% by weight acetyl zingerone; about 0.1-20% by weight azelaic acid; about 0.1-3.0% by weight bakuchiol; about 0.1-10.0% by weight licorice extract; about 0.1-25% by weight vitamin C or a derivative thereof, and a silicone based additive. In some embodiments, the composition further includes at least one of: polyethylene glycol (PEG)-30 dipolyhydroxystearate, polyglyceryl-3 diisostearate, diisopropyl adipate, ethoxydiglycol, water, ethylenediaminetetraacetic acid (EDTA), propylene glycol, glycerin, or xanthan gum. In some embodiments, the composition further includes at least one of: petrolatum, jojoba oil, polyglyceryl-3 diisostearate, diisopropyl adipate, dimethicone, or PEG-30 dipolyhydroxystearate. In some embodiments, the composition further includes about 1-10% by weight niacinamide. In some embodiments, the acetyl zingerone is about 0.2-1% by weight. In some embodiments, the acetyl zingerone is about 0.4-1% by weight. In some embodiments, the azelaic acid is about 1-15% by weight. In some embodiments, the azelaic acid is about 5-12% by weight. In some embodiments, the bakuchiol is about 1-2% by weight. In some embodiments, the licorice extract is about 1-7% by weight. In some embodiments, the licorice extract is about 2-6% by weight. In some embodiments, the vitamin C or a derivative thereof is about 1-12% by weight. In some embodiments, the vitamin C or a derivative thereof is about 4-12% by weight. In some embodiments, the silicone based additive is about 50-70% by weight. In some embodiments, the silicone based additive is about 55-65% by weight.

The present disclosure provides for a composition including: about 0.2-1% by weight acetyl zingerone; about 1-15% by weight azelaic acid; about 1-2% by weight bakuchiol; about 1-7% by weight licorice extract; about 1-12% by weight vitamin C or a derivative thereof, and a silicone based additive. In some embodiments, the composition further includes about 2-8% by weight niacinamide.

The present disclosure provides for a composition including: about 0.4-1% by weight acetyl zingerone; about 5-12% by weight azelaic acid; about 1-2% by weight bakuchiol; about 2-6% by weight licorice extract; about 4-12% by weight vitamin C or a derivative thereof; and a silicone based additive. In some embodiments, the composition further includes about 4-6% by weight niacinamide.

In some embodiments, the vitamin C or a derivative thereof is chosen from tetrahexyldecyl ascorbate, L-ascorbic acid, esters of ascorbic acid with fatty acids or their salts, ascorbyl palmitate, magnesium ascorbyl phosphate, sodium ascorbyl phosphate, ascorbyl glucoside, or 3-O-ethyl ascorbic acid. In some embodiments, the vitamin C or a derivative thereof is tetrahexyldecyl ascorbate.

The present disclosure provides for a composition including: about 0.1-2.0% by weight acetyl zingerone; about 0.1-20% by weight azelaic acid; about 0.1-3.0% by weight bakuchiol; and about 0.1-10.0% by weight licorice extract. In some embodiments, the composition further includes at least one of: polyethylene glycol (PEG)-30 dipolyhydroxystearate, polyglyceryl-3 diisostearate, diisopropyl adipate, ethoxy diglycol, water, ethylenediaminetetraacetic acid (EDTA), propylene glycol, glycerin, or xanthan gum. In some embodiments, the composition further includes at least one of: petrolatum, jojoba oil, polyglyceryl-3 diisostearate, diisopropyl adipate, dimethicone, or PEG-30 dipolyhydroxystearate. In some embodiments, the composition further includes about 1-10% by weight niacinamide. In some embodiments, the acetyl zingerone is about 0.2-1% by weight. In some embodiments, the acetyl zingerone is about 0.4-1% by weight. In some embodiments, the azelaic acid is about 1-15% by weight. In some embodiments, the azelaic acid is about 5-12% by weight. In some embodiments, the bakuchiol is about 1-2% by weight. In some embodiments, the licorice extract is about 1-7% by weight. In some embodiments, the licorice extract is about 2-6% by weight. In some embodiments, the composition further comprises about 0.1-25% by weight vitamin C or a derivative thereof. In some embodiments, the vitamin C or a derivative thereof is about 1-12% by weight. In some embodiments, the vitamin C or a derivative thereof is about 4-12% by weight.

In some embodiments, the present disclosure provides for a composition for use in treating an acne scar. In some embodiments, the present disclosure provides for a composition for use in treating hyperpigmentation. In some embodiments, the present disclosure provides for a composition for use in brightening skin. In some embodiments, the present disclosure provides for a composition for use in treating wrinkles on skin.

The present disclosure provides for a method for preparing a composition, the method including: mixing about 0.1-2.0% by weight acetyl zingerone, about 0.1-20% by weight azelaic acid, about 0.1-3.0% by weight bakuchiol, about 0.1-10.0% by weight licorice extract, and a silicone based additive to form a mixture; heating the mixture to 50-80 degrees Celsius; cooling the mixture to 40 degrees Celsius; combining the mixture with a ceramide complex including 1-5% ceramide to form a combination; and mixing the combination. In some embodiments, the composition further includes about 0.1-25% by weight vitamin C or a derivative thereof. In some embodiments, the composition is used as an acne scar gel.

The present disclosure provides for a method of treating a subject, the method including administering a composition to the subject, wherein the composition includes about 0.1-2.0% by weight acetyl zingerone, about 0.1-20% by weight azelaic acid, about 0.1-3.0% by weight bakuchiol, about 0.1-10.0% by weight licorice extract, and a silicone based additive, wherein the method reduces melanin levels in skin of a subject relative to an untreated subject, and wherein the composition is administered as a topical formulation. In some embodiments, the composition further includes about 0.1-25% by weight vitamin C or a derivative thereof.

In some embodiments, the present disclosure provides for a method, wherein the skin of the subject is lightened. In some embodiments, the present disclosure provides for a method, wherein the skin of subject has a reduction of dark spots. In some embodiments, the present disclosure provides for a method, wherein the skin of the subject is retextured. In some embodiments, the present disclosure provides for a method, wherein the skin of the subject has improved anti-inflammatory properties. In some embodiments, the present disclosure provides for a method, wherein the skin of the subject is protected against microbiomes.

### BRIEF DESCRIPTION OF DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows representative pictures of patients' facial skin at a baseline time and at 12 weeks after use of exemplary formulations of the present disclosure.

FIG. 2 is a graph representing patient perception of skin texture improvement at weeks 4, 8, and 12 (final follow-up week).

FIG. 3 is a graph representing patient perception of improvement in redness and/or pigmentation at weeks 4, 8, and 12 (final follow-up week).

### DETAILED DESCRIPTION

The following description and examples illustrate embodiments of the disclosure in detail. It is to be understood that this disclosure is not limited to the particular embodiments described herein and as such can vary. Those of skill in the art will recognize that there are numerous variations and modifications of this disclosure, which are encompassed within its scope.

### Definitions

Unless defined otherwise, all technical and scientific terms used herein have the same meaning as commonly understood by those of ordinary skill in the art to which embodiments herein belongs. Any methods, devices and materials similar or equivalent to those described herein can be used in the practice of embodiments herein. The following definitions are provided to facilitate understanding of certain terms used frequently herein and are not meant to limit the scope of the present disclosure. All documents (e.g., patent applications or patents) referred to herein are incorporated by reference in their entirety.

Herein, the use of the singular includes the plural unless specifically stated otherwise. It must be noted that, as used in the specification, the singular forms “a,” “an,” and “the” include plural referents unless the context clearly dictates otherwise.

As used herein, unless otherwise indicated, “about” or the like, when used in connection with a measurable variable (such as, for example, a parameter, an amount, a temporal duration, or the like), is meant to encompass variations of and from the specified value including those within experimental error (which can be determined by e.g., given data set, art accepted standard, and/or with e.g., a given confidence interval (e.g., 90%, 95%, or more confidence interval from the mean), such as variations of +/−20% or less, +/−10% or less, +/−5% or less, +/−1% or less, and +/−0.1% or less of and from the specified value, insofar such variations are appropriate to perform in the present disclosure. As used herein, the terms “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, and the like, and other factors known to those of skill in the art such that equivalent results or effects are obtained. In some circumstances, the value that provides equivalent results or effects cannot be reasonably determined. In general, an amount, size, composition, parameter or other quantity or characteristic is “about,” whether or not expressly stated to be such. It is understood that where “about,” is used before a quantitative value, the parameter also includes the specific quantitative value itself, unless specifically stated otherwise.

Herein, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Reference herein to “some embodiments,” “an embodiment,” “one embodiment,” or “other embodiments” means that a particular feature, structure, or characteristic described in connection with the embodiments can be included in at least some embodiments, but not necessarily all embodiments, of the present disclosure.

As used in this specification and claim(s), the words “comprising” (and any form of comprising, such as “comprise” and “comprises”); “having” (and any form of having, such as “have” and “has”); “including” (and any form of including, such as “includes” and “include”); or “containing” (and any form of containing, such as “contains” and “contain”) are inclusive or open-ended and do not exclude additional, un-recited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The terms, “decrease,” “reduce,” “reduction,” “lower,” “lowering,” or “inhibit” as used herein can generally mean a decrease by an amount which can be a statistically significant amount. For example, “decrease,” “reduce,” “reduction,” or “inhibit” can mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease, or any decrease between 10-100% as compared to a reference level.

The terms “increased,” “increase,” or “enhance” as used herein can generally mean an increase of an amount which can be a statistically significant amount; for the avoidance of doubt, the terms “increased,” “increase,” or “enhance,” can mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold, or at least about a 10-fold increase, or any increase between 2-fold and 10-fold or greater as compared to a reference level.

As used herein, the term “skin retexturing” refers to improving the appearance of the skin's surface appearance, for example by reducing the appearance of indented and/or raised scars and/or making skin appear smoother.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also, unless otherwise stated, include individual values (e.g., about 1%, about 2%, about 3%, about 4%, etc.) and the sub-ranges (e.g., about 0.5% to about 1.1%; about 0.5% to about 2.4%; about 0.5% to about 3.2%, about 0.5% to about 4.4%, and other possible sub-ranges, etc.) within the indicated range. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further disclosure. For example, if the value “about 10” is disclosed, then “10” is also disclosed.

### Overview

Skin is the first interface between the human body and its environment, that is, the interface between external and internal aggressions. A large variety of environmental factors such as solar ultraviolet (UV) light, visible light, environmental pollution, including ozone and particulate matter, increase the production of reactive oxygen species (ROS) in the skin. Apart from reactive species induced by exogenous sources within the skin itself, some ROS are also produced as oxidative metabolism byproducts in the mitochondria. Further, the skin microbiota compositions can be impacted by environmental factors such as UV and pollutants, which are also associated with skin health. Additionally, chronic psychological stress can also induce oxidative stress in the skin by lowering our antioxidant defenses. Oxidative stress and its resulting oxidative damages can exacerbate skin pigmentation and aging, inducing changes in skin complexion homogeneity, wrinkling, sagging, dryness and roughness.

Skin color is mainly determined by the amount and distribution of pigment molecules, and, to a lesser extent, it is also determined by the skin structure, which affects light scattering and impacts skin color. Melanin, a cluster of natural pigments originating from the epidermis, are synthesized by melanocytes through the process of melanogenesis. The abnormal production of melanin leads to dermatological problems, including melasma, post-inflammatory hyperpigmentation and actinic lentigo. The most commonly available skin depigmenting agents for commercial cosmetic use are tyrosinase inhibitors. Although a large number of tyrosinase inhibitors have been identified to date, only few of them have reached clinical applications as skin depigmenting agents because of safety concerns or weak effects. Many studies have reported that ROS and reactive nitrogen species (RNS) formation induced by UV can act as signaling messengers to elevate skin damage and melanogenesis while many antioxidants have been reported to show anti-pigmenting and anti-aging effects. Thus, an in-depth knowledge of the relationship between skin pigmentation, oxidative stress and antioxidant reactions may provide an acceptable rationale for developing unique formulations to achieve a more efficient method to prevent skin pigmentation and aging.

ROS has been shown to upregulate the matrix metalloproteinase-1 (MMP-1) enzyme, which chemically degrades collagen, whilst simultaneously inhibiting production of new collagen in a process that correlates with many of the hallmark signs of photoaging, including development of coarse and fine wrinkles as well as skin laxity. Antioxidant effectiveness was investigated using industry-standard chemical methods for measurement of radical scavenging against key ROS.

Skin aging and inflammation are critically linked. The inflammation-induced aging process involves a highly complex chain of events, by which acute inflammation gradually gives way to chronic or silent inflammation. It is this underlying inflammation that ultimately exhausts the body's defense system resulting in collagen and elastin degradation and the breakdown of the skin's barrier function. The inflammatory cascade of reactions that erode the skin's structure are ultimately deep wrinkles, hyperpigmentation and non-elastic tissues.

Propionibacterium acnes is a gram-positive human skin commensal that prefers anaerobic growth conditions and is involved in the pathogenesis of acne. Acne is one of the most common skin diseases, affecting more than 45 million individuals in the United States. The anaerobic bacterium Propionibacterium acnes is believed to play an important role in the pathophysiology of the common skin disease acne vulgaris. Over the last 10 years our understanding of the taxonomic and intraspecies diversity of this bacterium has increased tremendously, and with it the realization that strains are associated with skin health while others appear related to disease.

Compositions and formulations described herein can result in skin improvement targets. Formulations described herein comprise ingredients in amounts to provide synergistic effects. In some embodiments, formulations described herein are present in amounts to penetrate the skin. In some embodiments, formulations described herein are present in amounts to minimize skin irritation otherwise sometimes associated with the topical use of ingredients described herein.

### Active Agents

In some embodiments are compositions comprising one or more agents with properties to enhance skin status and/or skin condition. In some embodiments, the agent is an agent for treating hyperpigmentation. Exemplary agents for the treatment of hyperpigmentation include: kojic acid, azelaic acid, cysteamine, tranexamic acid, niacinamide, Vitamin C and its salts and esters or derivatives thereof, licorice extract, alpha-arbutin and extracts containing alpha-arbutin or other hydroquinone derivatives including but not limited to bearberry, hydroquinone and derivatives, resorcinol and derivatives, alpha and beta hydroxy acids, retinoids, bakuchiol, resveratrol, salicylic acid, and other peptides. In some embodiments, the agent is a retexturing agent. Exemplary retexturing agents include: dimethicone, cyclomethicone, dimethicone crosspolymers, alkyl dimethicones, dimethicone copolyol esters, silica, silicone elastomers, silicone resin, polymerized siloxanes, silicates, capric/caprylic triglyceride and other tryglycerides, jojoba oil, squalane, dimethyl isosorbide, fatty alcohols C₆-C₂₆ and esters, petrolatum, dimethicone copolyols, lanolin, natural butters and waxes, peptides, resveratrol, bakuchiol, growth factors, mineral oil, Vitamin C and its derivatives, yeast extract, and polyglyceryl esters. In some embodiments, the agent is an antioxidant. Exemplary antioxidants include: BHT, BHA, Vitamin C, Vitamin E, acetyl zingerone, propyl gallate, tannins, coenzyme Q10, ferulic acid and derivatives and analogs thereof. In some embodiments, the agent is for the treatment of acne. Exemplary agents for the treatment of acne include: bakuchiol, azelaic acid, resorcinol and derivatives thereof, salicylic acid, glycolic acid, and retinoids. In some embodiments, compositions described herein comprise a penetrating agent. Exemplary penetrating agents include: oleic acid (other similar fatty acids), water soluble glycols (propylene, butylene, pentylene, etc) and glycol ethers (e.g., ethoxydiglycol, ethoxytriglycol, butoxydiglycol, PEG, and PEG/PPG), dimethyl isosorbide, dimethyl sulfoxide, ethanol, lecithin and other phospholipids, liposomes, ethosomes, transfersomes, or niosomes. In some embodiments, compositions described herein comprise a stabilizing agent, or stabilizer. Exemplary stabilizing agents include: EDTA and salts thereof, sodium gluconate, acetyl zingerone, tetrasodium glutamate diacetate, alkyl dimethicone, phytic acid and salt derivatives, C₁₂-C₁₅ alkyl benzoate, castor oil, citric acid, or pH adjusters. In some embodiments, compositions described herein comprise a humectant. Exemplary humectants include: glycerin, hydrophilic glycols (C₃-C₆), hyaluronic acid and its salts, amino acids and amino acid derivatives, sodium lactate, sodium PCA, aloe, sorbitol, or beta-glucan. In some embodiments, compositions described herein comprise an emulsifier. Exemplary emulsifiers include: PEG-30 polyhydroxystearate, ethoxylated fatty alcohols, polysorbates, polyglyceryl-3 diisostearate, polyglyceryl esters, dimethicone copolyol and derivatives, PEG, PPG, PEG/PPG copolymers block polymers etc., modified polyacrylate and polyacrylamide, lecithin and other phospholipids, PEG/PPG esters, or PEG castor oil derivatives. In some embodiments, compositions described herein comprise a film former, or film-forming agent. Exemplary film formers include: dimethicone copolyols, dimethicone crosspolymers, or PEG diesters with polyhydroxystearic acid, polyglycerol esters (polyglyceryl-3 diisostearate, and polyglyceryl-2-triisostearate). In some embodiments, compositions described herein comprise an agent for the treatment of acne scars. Exemplary agents for the treatment of acne scars include: Dimethicone, Azelaic Acid, Tetrahexyldecyl Ascorbate, Ascorbic Acid, Diisopropyl Adipate, Glycerin, Ethoxydiglycol, Bakuchiol, PEG-30 Dipolyhydroxystearate, Capric/Caprylic Triglyceride, Glycyrrhiza glabra (Licorice Root) Extract, Hexylresorcinol, Ethyl Linoleate, Acetyl Zingerone, Tocopherol Acetate, or Polyglyceryl-3 Diisostearate. Exemplary silicone based additives in compositions of the present disclosure include: medical grade silicone gel, dimethicone, silicone elastomer, silicone resin, polymerized siloxane, silicate, silica, polysilicone-11, caprylyl methicone, cyclopentasiloxane, trisiloxane, dimethicone/vinyl dimethicone crosspolymer, PEG/PPG-19/19 dimethicone, PEG-12 dimethicone/PPG-20 crosspolymer, or any combinations thereof.

In some embodiments, the silicone based additive in compositions of the present disclosure comprise about 50-70% by weight of the composition. In some embodiments, the silicone based additive is present in about 55-70% by weight. In some embodiments, the silicone based additive is present in about 60-70% by weight. In some embodiments, the silicone based additive is present in about 65-70% by weight. In some embodiments, the silicone based additive is present in about 50-65% by weight. In some embodiments, the silicone based additive is present in about 50-60% by weight. In some embodiments, the silicone based additive is present in about 50-55% by weight.

In some embodiments are agents that provide skin protection and/or reversal of photodamage. In some embodiments, the agents can safeguard the skin's integrity more comprehensively. In some embodiments, compositions and formulations described herein comprise Acetyl Zingerone. Acetyl Zingerone is an agent with antiaging properties for use before or during the aging process. In some embodiments, acetyl zingerone is used to reverse visible signs of photo-damaged skin. In some embodiments, acetyl zingerone is associated with one or more of the following properties: maintenance of microbiome diversity, multifunctional antioxidant activity, oxygen radical quenching, oxygen radical quenching by non-sacrificial interaction, radical quenching of endogenous chromophores in the skin, iron chelation, diminished and/or prevention of Fenton reaction process(es), increased matrisome synthesis, a reduction in matrix metalloprotease (MMP) activity, reduction of intracellular reactive oxygen species (ROS) in keratinocytes exposed to UV-A light, inhibits or partially inhibits pro-inflammatory IL-17A cytokine responses. In some embodiments, acetyl zingerone is present in an amount of about 0.1-2.0% by weight. In some embodiments, acetyl zingerone is present in an amount of about 0.2-1% by weight. In some embodiments, acetyl zingerone is present in an amount of about 0.4-0.8% by weight.

In some embodiments, compositions and formulations described herein comprise Bakuchiol. Bakuchiol is an agent with collagen-stimulating properties. In some embodiments, bakuchiol is used to reverse visible signs of photo-damaged skin. In some embodiments, bakuchiol is used as an anti-aging agent. In some embodiments, bakuchiol is an alternative to retinol. In some embodiments, the use of bakuchiol results in wrinkle reduction and/or an increase in skin firmness. In some embodiments, bakuchiol is associated with one or more of the following properties: oxidative stress prevention of the skin, anti-inflammatory activity, inhibition of pro-inflammatory enzymes, a reduction in matrix metalloprotease (MMP) activity, maintenance of skin lipids, bacterial growth control, the correction of hyperkeratinization, and mitochondrial protection and the increase of ATP synthesis of the skin. In some embodiments, a composition described herein comprises bakuchiol in an amount measured by percent weight of about: 0.1 to 0.5, 0.5 to 1.0, 1.0 to 2.0, 2.0 2.5, or 2.5 to 3.0. In some embodiments, bakuchiol is present in an amount of about 0.1-3% by weight. In some embodiments, bakuchiol is present in an amount of about 1-2% by weight. In some embodiments, bakuchiol is present in an amount of about 1.5-2% by weight.

In some embodiments, compositions and formulations described herein comprise Azelaic acid. Azelaic acid is an agent with collagen-stimulating properties. In some embodiments, azelaic acid is used for the treatment of melasma, hyperpigmentation, rosacea, or a combination of any of these. In some embodiments, azelaic acid is an alternative to retinol. In some embodiments, the use of azelaic acid results in wrinkle reduction and/or an increase in skin firmness. In some embodiments, azelaic acid is associated with one or more of the following properties: anti-inflammatory activity, associated with decreased hyperpigmentation, anti-infective properties. In some embodiments, compositions are described herein comprise azelaic acid in an amount measured by percent weight of about: 0.1 to 0.5, 0.5 to 1.0, 1.0 to 2.0, 2.0 to 5.0, 5.0 to 8.0, 8.0 to 12.0, or 12.0 to 15.0. In some embodiments, azelaic acid is present in an amount of about 0.1-25% by weight. In some embodiments, azelaic acid is present in an amount of about 1-15% by weight. In some embodiments, azelaic acid is present in an amount of about 5-10% by weight.

In some embodiments, compositions and formulations described herein comprise ethyl linoleate and hexylresorcinol. Ethyl linoleate and hexylresorcinol are agents with anti-bacterial properties. In some embodiments, Ethyl linoleate and hexylresorcinol are used for the treatment of acne, a bacterial infection of the skin, or a combination of any of these. In some embodiments, Ethyl linoleate and hexylresorcinol are associated with one or more of the following properties: anti-inflammatory activity, decreased hyperpigmentation, anti-infective properties, regulation of follicular keratinization and barrier function, decreased sebaceous gland activity, anti-inflammatory activity by reducing NF-κB, reduced post-inflammatory hyperpigmentation (PIH), and reduced hyperpigmentation.

In some embodiments, compositions and formulations described herein comprise a plant-based extract. In some embodiments, the plant-based extract is a flavonoid. In some embodiments, the plant-based extract is licorice. In some embodiments, compositions and formulations described herein comprise licorice or an extract thereof. In some embodiments, compositions and formulations described herein comprise a licorice root extract. In some embodiments, compositions and formulations described herein comprise licorice. In some embodiments, compositions and formulations described herein comprise liquiritin. In some embodiments, compositions and formulations described herein comprise licochalcone A. Licorice is an agent associated with the minimization of tyrosinase production. Tyrosinase production is associated with skin discoloration. In some embodiments, licorice and is used to inhibit production of dark spots on the skin. In some embodiments, the dark spots are melanin spots. In some embodiments, licorice is associated with one or more of the following properties: associated with the prevention and/or decreased production of hyperpigmentation of the skin, anti-inflammatory properties, antioxidant properties, a decrease in ROS, anti-aging properties, decreased oil production in the skin, or a combination thereof. In some embodiments, the licorice extract is present in an amount of about 0.1-10% by weight. In some embodiments, the licorice extract is present in an amount of about 1-7% by weight. In some embodiments, the licorice extract is present in an amount of about 2-6% by weight.

### Exemplary Combinations of Agents for Improved Skin Properties

In some embodiments, compositions and formulations described herein comprise ethoxydiglycol. The use of ethoxydiglycol can suppress recrystallization of azelaic acid and Acetyl Zingerone and/or can enhance skin penetration for other agents in a composition or formulation described herein. The following description of particular embodiment(s) is merely exemplary in nature and is in no way intended to limit the scope of the disclosure, its application, or uses, which may, of course, vary.

In some embodiments, compositions described herein are for use in promoting lightening of the skin and/or reducing dark spots, reducing inflammation, improving skin retexturing, help protecting the skin's surface from oxidative damage caused by free radicals and environmental aggressors like UV light and pollution of skin, and also controlling the damaging skin microbiome. In some embodiments, azelaic acid is in the form of a compounded azelaic acid. In some embodiments, azelaic acid can be for use as an active melanin reducing agent.

In some embodiments, compositions described herein comprise an alpha-hydroxy acid. In some embodiments, compositions described herein comprise phytic acid. In some embodiments, the combination of agents described herein are present in a composition. In some embodiments, an agent described herein is present in a composition at a subclinical amount. In some embodiments, compositions described herein are associated with decreased irritation as compared to compositions with high acid concentrations (i.e., 20% azelaic acid and/or 10% phytic acid). In some embodiments, compositions are described herein comprise one or more hydroxy acids in amounts measured by percent weight of about: 0.1 to 0.5, 0.5 to 1.0, 1.0 to 2.0, 2.0 to 5.0, 5.0 to 8.0, 8.0 to 12.0, or 12.0 to 15.0. In some embodiments, compositions described herein comprise one or more hydroxy acids in amounts measured by percent weight of up to: 0.1, 0.2, 0.3, 0.4, 0.5, 0.6, 0.7, 0.8, 0.9, 1.0, 1.2, 1.5, 2.0, 2.5, 3.0, 3.5, 4.0, 4.5, or 5.0. In some embodiments, compositions are described herein comprise azelaic acid in an amount measured by percent weight of about: 0.1 to 0.5, 0.5 to 1.0, 1.0 to 2.0, 2.0 to 5.0, 5.0 to 8.0, 8.0 to 12.0, or 12.0 to 15.0. In some embodiments, compositions are described herein comprise phytic acid in an amount measured by percent weight of about: 0.1 to 0.5, 0.5 to 1.0, 1.0 to 2.0, 2.0 to 5.0, 5.0 to 8.0, or 8.0 to 10.0 by weight.

In some embodiments, are methods of treating hyperpigmentation with a composition described herein. In some embodiments, the composition comprises a 1,4-benzenediol, also referred to herein as “hydroquinone”. While effective, hydroquinone-based therapies have been shown to produce significant side effects of burning, redness, sensitization and irritation. Combining hydroquinone with other agents can decrease irritation and other side effects of the skin when administered. In some embodiments, a composition described herein comprises hydroquinone, phytic acid, and a hydroxy acid (e.g., azelaic acid). In some embodiments, the hydroxy acid is glycolic acid. In some embodiments, glycolic acid is optionally present. In some embodiments, the concentration of hydroquinone in a composition described herein is less than about 4% by weight. In some embodiments, the concentration of hydroquinone is less than about: 3.5%, 3.0%, 2.5%, 2.0%, 1.5%, 1.0%, 0.5%, 0.4%, 0.3%, 0.2%, or 0.1%.

In some embodiments, the concentration of a hydroxy acid in a composition described herein is optionally present in an amount measured by percent weight of about: 0.1 to 0.5, 0.5 to 1.0, 1.0 to 2.0, 2.0 to 5.0, 5.0 to 8.0, 8.0 to 12.0, 12.0 to 15.0, 15.0 to 18.0, 18.0 to 20.0, 20.0 to 22.0, or 22.0 to 25.0. In some embodiments, the concentration of azelaic acid in a composition described herein is optionally present in an amount measured by percent weight of about: 0.1 to 0.5, 0.5 to 1.0, 1.0 to 2.0, 2.0 to 5.0, 5.0 to 8.0, 8.0 to 12.0, 12.0 to 15.0, 15.0 to 18.0, 18.0 to 20.0, 20.0 to 22.0, or 22.0 to 25.0. In some embodiments, the concentration of phytic acid in a composition described herein is optionally present in an amount measured by percent weight of about: 0.1 to 0.5, 0.5 to 1.0, 1.0 to 2.0, 2.0 to 5.0, or 5.0 to 8.0. In some embodiments, Azelaic acid and phytic acid are present as the only skin lightening agents present in a composition described herein. In some embodiments, phytic acid and glycolic acid are present as the only skin lightening agents present in the composition.

In some embodiments, azelaic acid is optionally present in a composition in amounts from 0.1% to 15% by weight or any value or range therebetween. In some embodiments, azelaic acid is present from 1.0% to 10% by weight. In some embodiments, azelaic acid is present from 1.0% to 5% by weight. Optionally, azelaic acid is present at 2.0% by weight. In some embodiments, the azelaic acid is in solution. In some embodiments, the azelaic acid is partially dissolved. In some embodiments, the azelaic acid is entirely dissolved.

In some embodiments, hydroxy acid is present from 1.0% to 10% by weight. In some embodiments, hydroxy acid is present from 1.0% to 5% by weight. Optionally, hydroxy acid is present at 2.0% by weight. Glycolic acid is an exemplary hydroxy acid that is optionally present at any of the aforementioned levels or ranges of levels.

In some embodiments, compositions described herein comprise Azelaic acid and a hydroxy acid, (e.g., glycolic acid) at amounts of 0.1 to 15 percent by weight each, optionally 1.0% to 10% by weight each, optionally 1.0% to 5% by weight each. Optionally, both Azelaic acid and glycolic acid are present at 2% by weight. In some embodiments, phytic acid is present in a composition at 0.1% and 10.0% by weight or any value or range therebetween. In some embodiments, phytic acid is present at 1.0% to 5.0% by weight. Optionally, phytic acid is present at 0.5% to 5% by weight. Optionally, phytic acid is present at 2.0% by weight.

Compositions described herein can comprise secondary ingredients and/or additional materials including emollients, thickeners, carriers, or vehicles, so as to form a liquid, gel, or cream suitable for topical application to the skin of a subject. For example, the secondary ingredients may include buffers/pH adjusters, dyes/colorants, moisturizers, fragrances, vitamins, texture modifiers, essential oils, and anti-microbial agents, combinations thereof, or the like. In various embodiments, the compositions may include from about 0 wt % to about 8 wt % secondary ingredients.

In some embodiments, a composition optionally includes a pH-adjusting agent. Illustratively, a pH adjusting agent is triethanolamine. Triethanolamine associates with one or more acids present in the composition forming salts. The salts are less irritating than the free acids. The presence of triethanolamine in the composition also serves to raise the pH to a more topically acceptable level. It is appreciated that other pH-adjusting agents suitable to increase the pH relative to a composition without a pH-adjusting agent are operable in a composition. Such pH-adjusting agents are known in the art. The buffers/pH adjusters may include calcium ions, potassium ions, or hydroxide ions, any combination thereof, or any salts or compounds capable of generation such ions. The buffers/pH adjusters may be blended at various ratios, in order to provide a suitable pH. In some embodiments, the secondary ingredients may include essential oils such as lavender oil, rosemary oil, cedar wood oil, thyme oil, peppermint oil, chamomile oil, sage oil, lemon oil, patchouli oil, tea tree oil, ylang ylang oil, vetiver oil, carrot seed oil, cypress oil, helichrysum oil, combinations thereof, or the like. In various embodiments, a suitable fragrance may be used. Useful fragrances may be in liquid form, such as traditional fragrances that are combinations of synthetic and natural compounds, natural fragrances that consist of a blend of natural extracts and essential oils, or essential oils in the pure and neat form.

A composition is optionally an aqueous composition defined herein as 40% or greater water. Water is optionally purified so as to remove contaminants such as solids and other microorganisms, or subjected to processes to remove contaminating ions. Illustratively, compositions include deionized water prepared by methods and using apparatuses known in the art. Methods of purifying or filtering water are well known in the art. It is appreciated that some embodiments of the composition are anhydrous.

A composition optionally includes one or more non-aqueous solvents such as an alcohol. Illustratively, a solvent is ethanol, isopropyl alcohol, methanol, ethoxydiglycol, benzyl alcohol, polyethylene glycol, dimethylisosorbide, triacetin (glyceryl triacetate), butylene glycol, propylene glycol, hexylene glycol, or other appropriate solvent as recognized in the art. In some embodiments, an alcohol is ethanol. Optionally, ethanol is specially denatured ethanol. An illustrative example of ethanol is SD alcohol 40-B. Alcohol is optionally present at 5% to 99.8% by weight, optionally 10% to 45% by weight. Optionally, alcohol is present at 20% to 30% by weight. In some embodiments, the composition is anhydrous.

A composition optionally includes one or more hydroxy acids. Examples of hydroxy acids illustratively include: beta-hydroxy acids illustratively salicylic acid, acetylsalicylic acid, among others; or alpha-hydroxy acids illustratively mandelic acid, glycolic acid, lactic acid, tartaric acid, malic acid, and citric acid, among others. Optionally, a composition includes mandelic acid as the sole hydroxy acid. A hydroxy acid is optionally preset at an amount of 0.1% to 20% by weight. Optionally, a hydroxy acid is present at from 1.0% to 10% by weight or any value or range therebetween. Optionally, a hydroxy acid is mandelic acid preset at from 1.0% to 5% by weight, optionally at 3.0% by weight. Optionally, a hydroxy acid is glycolic acid preset at from 1.0% to 5% by weight, optionally at 3.0% by weight. Optionally, both mandelic acid and glycolic acid are present. Optionally, glycolic acid is present absent mandelic acid. In some embodiments, one or more hydroxy acids are present and Azelaic acid is absent.

A composition optionally includes one or more additives. It is appreciated, however, that a composition is optionally free of an additive. An additive illustratively is one or more antioxidants, antiperspirants, anti-static agents, buffering agents, bulking agents, chelating agents, cleansers, colorants, conditioners, diluents, dyes, emollients, flavonoids, fragrances, humectants, ionization agents, moisturizers, occlusive agents, perfuming agents, pearlescent aids, perfuming agents, permeation enhancers, pH-adjusting agents, preservatives, protectants, skin penetration enhancers, softeners, solubilizers, sunscreens, sun blocking agents, sunless tanning agents, or viscosity modifiers. Optionally, a composition is free of 1,2-decanediol. The source and type of additive operable herein is readily understood by one of skill in the art.

In some embodiments, a humectant is optionally included in a composition. Illustrative examples of humectants include: glycerin, glycereth-7 trimethyl ether, propylene glycol and propylene glycol derivatives, guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, alkoxylated glucose, hyaluronic acid, salts of hyaluronic acid, lactamide monoethanolamine, acetamide monoethanolamine and derivatives, esters, salts and mixtures thereof, as well as any suitable humectant found in Handbook of Pharmaceutical Additives published by Gower where one of ordinary skill in the art will recognize suitable humectants contained therein. A humectant, when present, is optionally present at 1.0% to 15% by weight, optionally at 5.0% to 10% by weight. In some embodiments, a humectant is glycereth-7 trimethyl ether present at 5% to 10% by weight, optionally 7.0% by weight.

Some embodiments of a composition include one or more anti-irritants. Illustrative examples of anti-irritants include materials derived from plants such as plant extracts or plant juices. An anti-irritant is optionally a plant extract, illustratively, Anthemis nobilis flower extract, bisabolol, alpha-bisabolol, Arctium lappa, Boerhavia diffusa root extract, Echinacea, among other plant extracts known in the art. Other non-limiting examples of an anti-irritant include calcium gluconate, coenzyme Q10, among others known in the art. An anti-irritant is optionally present in a composition at from 0.001% to 2.0% by weight.

A composition is optionally provided as a serum, lotion, cream, gel, bar, ointment, in pad form, or other desirable form for topical administration to a subject. Optionally, the composition is supplied in the form of an aqueous solution. Compositions described herein optionally have a pH in the range between 2.0 and 6.0 or any value or range therebetween. A pH of the composition is optionally from 3.0 to 4.5, optionally 3.0 to 4.0, optionally 3.5 to 4.5, optionally 3.5 to 4.0.

In some embodiments, a composition described herein optionally includes one or more non-aqueous solvents such as a silicone or ester. Illustratively, a solvent is ethanol, isopropyl alcohol, ethoxytriglycol, ethoxydiglycol, isostearyl isostearate, polyethylene glycol, dimethylisosorbide, diisopropyl adipate, butylene glycol, propylene glycol, hexylene glycol, cyclomethicone, or other appropriate solvent as recognized in the art. In some embodiments, a cyclomethicone is cyclopentasiloxane. Optionally, cyclomethicone is cyclohexasiloxane. An illustrative example of cyclomethicone is DOW PMX 245. Cyclomethicone is optionally present at 5% to 99.8% by weight, optionally 10% to 45% by weight. Optionally, cyclomethicone is present at 20% to 30% by weight. In some embodiments, the composition is anhydrous. In some embodiments, a composition described herein comprises medical grade silicone gel.

A composition optionally includes one or more retexturing agents. Examples of retexturing agents include retinoids, bakuchiol, vitamin C and its derivatives, live yeast cell derivatives, polyglyceryl esters, silica, silicone elastomers, silicone resin, dimethicone, dimethicone crosspolymers, alkyl dimethicones, dimethicone copolyols, dimethicone copolyol esters, jojoba oil, squalene, dimethyl isosorbide, fatty alcohols and esters C₁₂-C₂₆, petrolatum, mineral oil, resveratrol, Azelaic acid, and peptides.

A composition optionally includes one or more vitamins. A vitamin is illustratively vitamin A or its derivatives, vitamin C or its derivatives, vitamin E or its derivatives, vitamin D, vitamin B2, vitamin B3, vitamin B5, vitamin B6, vitamin B8, vitamin B9, or vitamin B12. Optionally, a vitamin A derivative is retinal, retinoic acid, retinyl ester, retinol, tretinoin, isotretinoin, adapalene, tazarotene, or combinations thereof. Vitamin A or a derivative thereof is optionally present at between 0.001 to 5 weight percent. Vitamin C is optionally present as L-ascorbic acid. Vitamin C derivatives are optionally esters of ascorbic acid with fatty acids or their salts. An illustrative ester of ascorbic acid with fatty acids is ascorbyl palmitate. In some embodiments, the Vitamin C is tetrahexyldecyl ascorbate. In some embodiments, the Vitamin C is L-ascorbic acid. In some embodiments, the Vitamin C is chosen from esters of ascorbic acid with fatty acids or their salts. In some embodiments, the Vitamin C is ascorbyl palmitate. In some embodiments, the Vitamin C is magnesium ascorbyl phosphate. In some embodiments, the Vitamin C is sodium ascorbyl phosphate. In some embodiments, the Vitamin C is ascorbyl glucoside. In some embodiments, the Vitamin C is or 3-O-ethyl ascorbic acid. Vitamin C is optionally present at 0.1% to 35%. In some embodiments, Vitamin C is present in an amount of about 0.1-25% by weight. In some embodiments, Vitamin C is present in an amount of about 1-10% by weight. In some embodiments, Vitamin C is present in an amount of about 4-8% by weight. A vitamin is optionally vitamin B3 (niacinamide, niacin, or nicotinic acid). Vitamin B3 is optionally present at 1.0 to 10% by weight, optionally 1.0% to 5.0% by weight, optionally at 3.0% by weight. A vitamin is optionally niacinamide present at 1.0% to 5.0% by weight, optionally at 3.0% by weight. In some embodiments, a composition is free of all vitamins with the exception of niacinamide. In some embodiments, the compositions comprise 1-10% niacinamide. In some embodiments, the compositions comprise 2-8% niacinamide. In some embodiments, the compositions comprise 4-6% niacinamide.

In some embodiments, compositions described herein comprise an antioxidant. Illustrative examples of antioxidants include acetyl zingerone, vitamin C and its derivatives, BHT, BHA, vitamin E and its derivatives, ferulic acid and its derivatives, propyl gallate, coenzyme Q10, tannins, and flavonoids, among others. An antioxidant, when present, is optionally present at 0.1-15%.

In some embodiments, compositions described herein comprise a combination of Acetyl Zingerone and C-Tetrahexyldecyl Ascorbate (THDC) is an L-ascorbic acid precursor with improved stability and ability to penetrate the epidermis. Acetyl zingerone is an effective antioxidant stabilizer of THDC and that combination of these products may improve ascorbic acid delivery. This provides a step towards reaching the full potential of ascorbate as an active ingredient in topical preparations.

In some embodiments, the composition of the present disclosure comprises (in weight percentage of the final product): 1-3% PEG-30 dipolyhydroxystearate, 0.25-3% polyglyceryl-3 diisostearate, 2-20% diisopropyl adipate, 0.5-5% ethoxydiglycol, 0.25-1.5% acetyl zingerone, 3-15% DOW 11-030, 3-15% DOW EL-7040, 2-21% cyclopentasiloxane, 2-21% DOW PMX 1184, 2-20% DOWSIL 9701, 0.1-25% azelaic acid, 0.1-3% bakuchiol, 0.1-1.5% vitamin E acetate, 0.25-3% capric/caprylic triglyceride, 0.25-1.5% hexylresorcinol, 0.25-1.5% ethyl linoleate, 3-15% tetrahexyldecyl ascorbate, 0.1-10% licorice extract, and 5-30% gransil DM-5 AA 30LV.

In some embodiments, the composition of the present disclosure comprises (in weight percentage of the final product): 1-2% PEG-30 dipolyhydroxystearate, 1-2% polyglyceryl-3 diisostearate, 4-10% diisopropyl adipate, 1-3% ethoxydiglycol, 0.4-1% acetyl zingerone, 3-15% DOW 11-030, 5-10% DOW EL-7040, 5-15% cyclopentasiloxane, 5-15% DOW PMX 1184, 5-10% (5.5%) DOWSIL 9701, 5-15% azelaic acid, 1.5-2% bakuchiol, 0.2-1% vitamin E acetate, 1-2% capric/caprylic triglyceride, 0.4-1% hexylresorcinol, 0.4-1% ethyl linoleate, 4-10% tetrahexyldecyl ascorbate, 2-8% licorice extract, and 10-20% gransil DM-5 AA 30LV.

In some embodiments, the formulation of the present disclosure comprises (in weight percentage of the final product) 40-80% water, 0.01-0.5% EDTA, 0.05-0.5% gellan gum, 3-25% diisopropyl adipate, 0.25-2% lecithin, 0.1-25% azelaic acid, 1-10% C₁₂₋₁₈ alkanoyl glycerin/sebacic acid copolymer, 0.5-4% PEG-100 stearate, 0.5-4% glyceryl stearate, 0.5-5% stearyl alcohol, 0.1-5% ethoxydiglycol, 0.05-1.5% acetyl zingerone, 0.1-3% bakuchiol, and 0.1-25% tetrahexyldecyl ascorbate.

In some embodiments, the formulation of the present disclosure comprises (in weight percentage of the final product) 40-60% water, 0.01-0.2% EDTA, 0.1-0.3% gellan gum, 10-20% diisopropyl adipate, 0.4-0.8% lecithin, 5-15% azelaic acid, 2-5% C₁₂₋₁₈ alkanoyl glycerin/sebacic acid copolymer, 1-2% PEG-100 stearate, 1-2% glyceryl stearate, 1-3% stearyl alcohol, 2-3% ethoxydiglycol, 0.2-0.8% acetyl zingerone, 0.5-2% bakuchiol, and 5-15% tetrahexyldecyl ascorbate.

In some embodiments, the formulation of the present disclosure comprises (in weight percentage of the final product) 40-80% water, 0.05-1.5% hyaluronic acid, 0.5-3% propylene glycol, 1-5% glycerin, 0.1-1.5% polysorbate-80, 0.05-0.2% EDTA, 0.1-0.5% xanthan gum, 1-10% tranexamic acid, 2-10% niacinamide, 2-10% licorice extract, 0.1-2% panthenol, 1-10% petrolatum, 1-7% jojoba oil, 0.25-3% polyglyceryl-3 diisostearate, 1-8% diisopropyl adipate, 1-5% dimethicone, 0.25-3% PEG-30 dipolyhydroxystearate, 0.25-3% bakuchiol, and 1-5% ceramide complex.

In some embodiments, the formulation of the present disclosure comprises (in weight percentage of the final product) 40-60% water, 0.5-1.5% hyaluronic acid, 0.5-1.5% propylene glycol, 2-3% glycerin, 0.5-1% polysorbate-80, 0.05-0.15% EDTA, 0.1-0.3% xanthan gum, 2-8% tranexamic acid, 2-6% niacinamide, 5-10% licorice extract, 0.5-1.5% panthenol, 1-5% petrolatum, 4-7% jojoba oil, 1-2.5% polyglyceryl-3 diisostearate, 2-5% diisopropyl adipate, 2-4% dimethicone, 2-2.5% PEG-30 dipolyhydroxystearate, 0.5-2% bakuchiol, and 2-4% ceramide complex.

### Methods for Use

Also provided are processes of reducing melanin levels or a portion thereof, in a subject, optionally in the skin of a subject. A process includes administering a therapeutically effective amount of a composition as described herein, and equivalents thereof, to the skin of a subject, or to a cell, and reducing the level of melanin in the skin or cell by the administering a therapeutically effective amount is that sufficient to reduce the level of melanin on the tissue onto which the composition is applied.

In some embodiments, a composition is administered to a subject once or more daily until the desired level of melanin decrease is achieved. A composition is optionally administered 1, 2, 3, 4, 5, or more times daily. Administration is optionally 1, 2, 3, 4, 5, 6, 7, or more times per week. Administration is optionally continued until a desired amount of melanin decrease is achieved. A composition is optionally administered once. A composition is optionally administered for 1, 2, 3, 4, 5, 6, 7, 8, 9, 10, 11, 12, or more weeks.

In some embodiments, a composition is optionally administered to a subject and allowed to incubate in contact with the subject for a desired incubation time followed by removal such as by washing or wiping with a cloth. A composition is optionally incubated on the skin for 1, 2, 3, 4, 5, 6, 7, 8, 9, 10, or more minutes. In some embodiments, a composition is allowed to remain in contact with the subject indefinitely without conscious or intentional removal.

In some embodiments, a composition described herein is suitable for reducing melanin content of the skin, and use of the composition for promoting lightening of the skin and reducing dark spots, reducing inflammation, improving skin retexturing, protecting the skin's surface from oxidative damage caused by free radicals and environmental aggressors like UV light and pollution of skin, and also maintaining a healthy skin microbiome. It was surprisingly discovered that, contrary to the art recognized requirement of relatively high levels of these agents for skin lightening, combining Azelaic acid with bakuchiol or vitamin C at sub-clinically effective levels provides synergistic and improved skin lightening properties with greatly reduced irritation.

Also provided are processes of reducing melanin levels or a portion thereof in a subject, optionally in the skin of a subject. In some embodiments, the process of reducing melanin levels comprises: administering a therapeutically effective amount of a composition as described herein, and equivalents thereof, to the skin of a subject, or to a cell, and reducing the level of melanin in the skin or cell by the administering a therapeutically effective amount is that sufficient to reduce the level of melanin on the tissue onto which the composition is applied.

In some embodiments are compositions for the management and/or treatment of post-acne scaring. In some embodiments are compositions for the management and/or treatment of hyperpigmentation. In some embodiments are compositions for the management scar appearance.

In some embodiments are compositions for concurrent use with a sunscreen.

### Formulations

Also described herein formulations of the present disclosure can be prepared by adding the components of the below examples and the different phases to a suitable vessel and heating while stirring until a homogeneous melt is obtained. The composition can be cooled and added to the other phases. If heat sensitive ingredients are to be used, they may be dispersed into the certain phases after cooling. In some embodiments, the composition is mixed by high shear mixing to adequately disperse the ingredients and agents.

Also described herein are formulations comprising compositions described herein. In some embodiments, formulations described herein comprise at least one of: azelaic acid, vitamin C, bakuchiol, or others. In some embodiments, the formulation is a liposomal formulation. In some embodiments, the composition is formulated into liposomes. Liposomes are widely used vehicles for drug delivery, owing to their proven biocompatibility, biodegradability, and ability to encapsulate both hydrophilic and hydrophobic compounds. Liposome sizes can range from 0.1 micron to 1000 micron. Liposomes are vesicles in which an aqueous volume (which can encapsulate hydrophilic compounds) is enclosed by a spherical lipid bilayer (which can encapsulate hydrophobic compounds) typically composed of phospholipids and additional agents Phospholipids, as the main component of liposomes, are amphiphilic molecules, composed of a polar head and a nonpolar tail. When the drug is hydrophilic, it will be entrapped in the core (interior) of the liposome vesicles. Conversely, a hydrophobic molecule will be encapsulated in the middle of the lipid bilayer that is constructed of nonpolar tails of the phospholipid. Liposomes can be absorbed onto the skin and fuse with the lipid bilayer of the stratum corneum (SC) of the skin, which resulting in the disruption of the outer layer integrity. Therefore, the lipids act as skin penetration enhancers that facilitate drug permeation into skin. Liposomes have been used for the delivery of various different kinds of drugs and ingredients, such as diclofenac baicalein, amphotericin B, ketoprofen, vitamin C and azithromycin. Formulations described herein can include a blend of liposomes for different ingredients with different liposome sizes. Liposomes can prevent active agents in compositions described herein from interacting with each other and also may reduce degradation.

### EXAMPLES

### Example 1: Exemplary Formulations for Skin Improvement

TABLE 1
Exemplary Formulation I

Phase A: (weight % of final product)
PEG-30 dipolyhydroxystearate 1-3%
Polyglyceryl-3 diisostearate 0.25-3%
Diisopropyl adipate 2-20%
Ethoxydiglycol 0.5-5%
Acetyl zingerone 0.25-1.5%
Phase B (weight % of final product)
PEG/PPG-19/19 Dimethicone 2-10%
Caprylyl methicone 2.5-12%
PEG-12 Dimethicone/PPG-20 Crosspolymer 0.25-3%
Cyclopentasiloxane 2-21%
Dimethicone 1-12%
Trisiloxane 0.5-10%
Phase C (weight % of final product)
Dimethicone/Vinyl Dimethicone Crosspolymer and Silica 2-20%
Azelaic Acid 0.1-20%
Phase D (weight % of final product)
Bakuchiol 0.1-3%
Vitamin E Acetate 0.1-1.5%
Capric/caprylic triglyceride 0.25-3%
Hexylresorcinol 0.25-1.5%
Ethyl linoleate 0.25-1.5%
Tetrahexyldecyl ascorbate 3-15%
Phase E (weight % of final product)
Licorice extract 0.1-10%
Phase F (weight % of final product)
Ascorbic acid 1.5-10%
Polysilicone-11 0.25-8%
Dimethicone 4-20%

### Procedure:

As listed in Table 1, Phase A ingredients were mixed together and heated to 50 degrees C. while mixing. In a separate vessel, Phase B ingredients were mixed together. The Phase A ingredients mixture was cooled to room temperature and mixed with the Phase B ingredients mixture. Phase C ingredients were slowly added to the Phase B mixture, one ingredient at a time. As the viscosity got too high to mix while the Phase C ingredients were being added, Phase D ingredients were slowly added, one ingredient at a time, alternating between the Phase C and Phase D ingredients. Phase E ingredients were added and mixed in with a high shear. Finally, Phase F ingredients were added and mixed in.

TABLE 2
Exemplary Formulation II

Phase A: (weight % of final product)
Water 40-80%
EDTA 0.01-0.5%
Gellan gum 0.05-0.5%
Phase B: (weight % of final product)
Diisopropyl adipate 3-25%
Lecithin 0.25-2%
Azelaic acid 0.1-20%
Phase C: (weight % of final product)
C12-18 Alkanoyl Glycerin/Sebacic Acid Copolymer 1-10%
PEG-100 stearate 0.5-4%
Glyceryl stearate 0.5-4%
Stearyl alcohol 0.5-5%
Dimethicone 0.25-5%
Cyclopentasiloxane 0.5-10%
Phase D: (weight % of final product)
Ethoxydiglycol 0.1-5%
Acetyl zingerone 0.05-1.5%
Bakuchiol 0.1-3%
Tetrahexyldecyl ascorbate 0.1-25%
Licorice extract 2-6%

### Procedure:

As listed in Table 2, Phase A ingredients were mixed together and heated to 75 degrees C. while mixing. The mixture was held at 75 degrees C. In a separate vessel, diisopropyl adipate and lecithin were heated to 95 degrees C. while mixing. Once at 95 degrees C., Azelaic acid was slowly added to the rest of the phase B ingredients and mixed. Once the azelaic acid was dissolved, the Phase C ingredients were added to the Phase B mixture, and mixed while maintaining the temperature. Phases B and C were added to the Phase A mixture and were mixed with a high shear at 75 degrees C. for 30 minutes. The mixture comprising the ingredients of Phases A to C were allowed to cool. Phase D ingredients were mixed together in a separate vessel and heated to 45 degrees C. Once the mixture comprising Phases A to C were below 35 degrees C., the Phase D mixture was added.

TABLE 3
Exemplary Formulation III

Phase A: (weight % of final product)
Water 40-80%
Hyaluronic acid 0.05-1.5%
Propylene glycol 0.5-3%
Glycerin 1-5%
Polysorbate-80 0.1-1.5%
EDTA 0.05-0.2%
Xanthan gum 0.1-0.5%
Phase B: (weight % of final product)
Tranexamic acid 1-10%
Niacinamide 2-10%
Licorice extract 2-10%
Panthenol 0.1-2%
Phase C: (weight % of final product)
Petrolatum 1-10%
Jojoba oil 1-7%
Polyglyceryl-3 diisostearate 0.25-3%
Diisopropyl adipate 1-8%
Dimethicone 1-5%
PEG-30 dipolyhydroxystearate 0.25-3%
Bakuchiol 0.25-3%
Phase D: (weight % of final product)
Ceramide complex 1-5%

### Procedure:

As listed in Table 3, Phase A ingredients were mixed together and heated to 80 degrees C. Phase C ingredients were mixed together in a separate vessel and heated to 65 degrees C. After 15 minutes, Phase A was cooled to 65 degrees C. and Phase B ingredients were added, one ingredient at a time to the Phase A mixture. Phases A, B, and C were mixed together at 65 degrees C., at a high shear for 30 minutes and then allowed to cool down. Finally, Phase D ingredients were mixed in at 40 degrees C.

### Example 2: Treatment with Exemplary Formulations

Human subjects were observed for twelve weeks with 4 visits to the study site where they were each evaluated while using an exemplary formulation described herein. Acne scarring of each subject was monitored with 3D imaging using Quantificare 3-D Life-Viz camera. Evaluation of the improvement in hyperpigmentation of each subject was then assessed using the Taylor hyperpigmentation Scale.

### Example 3: Exemplary Formulations for Improving Acne Scar Appearance and Reducing Hyperpigmentation

An acne scar gel (ASG) comprising exemplary Formulation I, as described above, was prepared as a non-invasive topical therapy for acne scarring. A single-center, open label, study was conducted to evaluate the effectiveness of the ASG in improving acne scar appearance and reducing hyperpigmentation in subjects with mild to moderate acne scars.

### Study Methods

The efficacy of ASG was examined in a single center, open label clinical trial involving otherwise healthy subjects (n=27, age range 18 to 45 years) presenting with mild to moderate acne scarring and dormant acne. A minimum of 60% of the subject group was to present with hyperpigmentation. The study was conducted under good clinical practice guidelines and informed consent was obtained from all subjects as per the Declaration of Helsinki.

Exclusion criteria included (but was not limited to) presence of any systemic disorder or face dermatoses other than acne that would in any way confound interpretation of the study results (e.g., atopic dermatitis, eczema, or psoriasis); history of skin cancer; female subjects who are pregnant, expect to become pregnant, or are lactating; subjects who have started, ceased, or changed hormonal therapy of any kind within 3 months prior to the study; actual or planned excessive exposure to natural or artificial UV light (such as tanning, phototherapy, or a tropical vacation) within 1 month prior to the study; use of systemic drugs for more than 3 consecutive days related to antibiotics, anti-inflammatory, or corticoids 4 weeks prior, or and/or use of topical drugs for more than 3 consecutive days related to antibiotics, anti-inflammatory, or corticoids in the 2 weeks prior to the study; or the presence of any condition or use of medication and/or history of medical/surgical events which, in the opinion of the investigator, could compromise the safety of the subject or affect the outcome of the study. Additionally, use of the ASG is contraindicated in patients with a known sensitivity to silicone, vitamin C, bakuchiol or azelaic acid.

Study duration was 4 visits over a 12-week period at baseline (week 0) and follow-up at weeks 4, 8, and final follow-up at week 12. At the baseline visit subjects were given an adequate supply of ASG (as well as an SPF in case of sun exposure) and instructed on the proper use of the products, as well as given a reference sheet to take home regarding ASG use during the study. ASG was to be applied twice daily, once before bedtime and again in the morning, to all areas with acne scarring. When applying the morning dose of ASG, if the subject planned to be exposed to the sun, they were instructed to wait 10 minutes, and then apply the SPF.

The primary evaluation was 3D imaging photography with randomized blinded observer evaluation of the photographs. Hyperpigmentation was measured using the Taylor Hyperpigmentation Scale. (Taylor et al., Cutis. 2005, 76(4):270-4).

A secondary patient questionnaire was also administered (subjective rating 1-5 where 1=disagree, 2=slightly disagree, 3=neither agree nor disagree, 4=slightly agree, 5=agree) with 10 questions about hyperpigmentation and skin appearance (scores added for a total between 10 and 50) and 6 questions focused on impressions of the product and its use (score totals not relevant). These evaluations were performed at weeks 4, 8 and 12 (final follow-up).

### Results

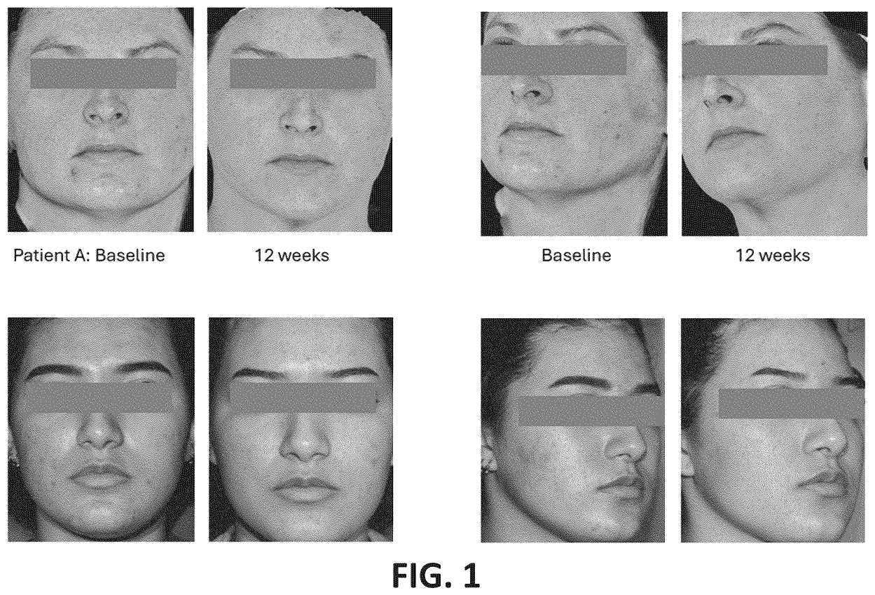

No significant treatment-related adverse events were noted. Of 27 subjects, 24 completed the study and 3 were lost to follow-up. Preliminary data shows that approximately 80% of subjects had smoother skin and reduced hyperpigmentation and redness at the final week 12 follow-up visit. FIG. 1 shows representative pictures of patient's facial skin at baseline and after 12 weeks of the study using ASG. The pictures were assessed by three independent observers: (1) an physician with over thirty-five years of experience, (2) a physician with over eleven years of experience, and (3) a physician with over eight years of experience. Physician (1) correctly identified 16 of 24 before/after pictures (i.e., 66.67%); Physician (2) correctly identified 21 of 24 before/after pictures (i.e., 87.5%); and Physician (3) correctly identified 13 of 24 before/after pictures (i.e., 66.67%). The overall observance was 50 of 72 before/after pictures correctly identified (i.e., 69.44%).

Subjective questionnaire results showed high patient satisfaction and overall positive impressions of the product itself and its regular use. Patient perception of skin texture and coloration throughout the study duration was also rated via subjective questionnaire, and the results were averaged at each time point; total scores were also calculated for the 10 questions relating to skin improvement, and the 6 questions relating to product use. Improvement was indicated by increases in average score from one time point rating to another, later time point.

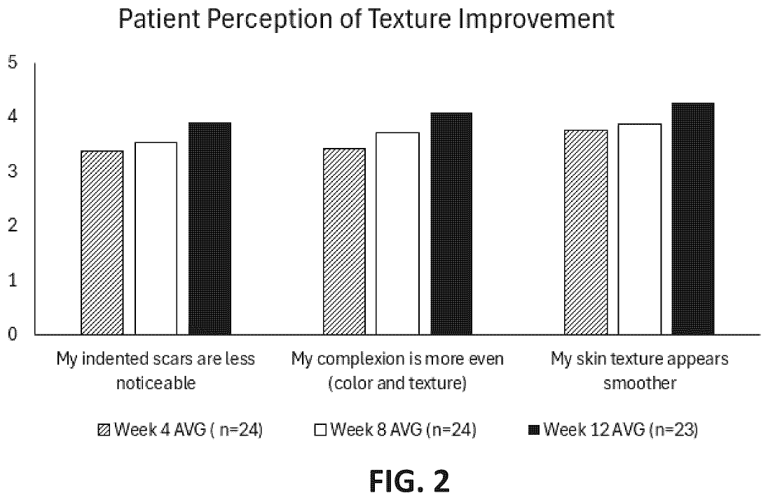

For skin texture and coloration, aggregate scores (possible totals ranging between 10 and 50) for weeks 4, 8, and 12 were 34.17, 35.33, and 38.91, respectively. This denotes an average increase of 1.16 between weeks 4 and 8 (approximately 3.4%), an average increase of 3.58 between weeks 8 and 12 (approximately 10.1%), and an average increase of 4.74 between weeks 4 and 12 (approximately 12.2%). FIG. 2 of the accompanying drawings provides a visual representation of these changes for three key questions in this category.

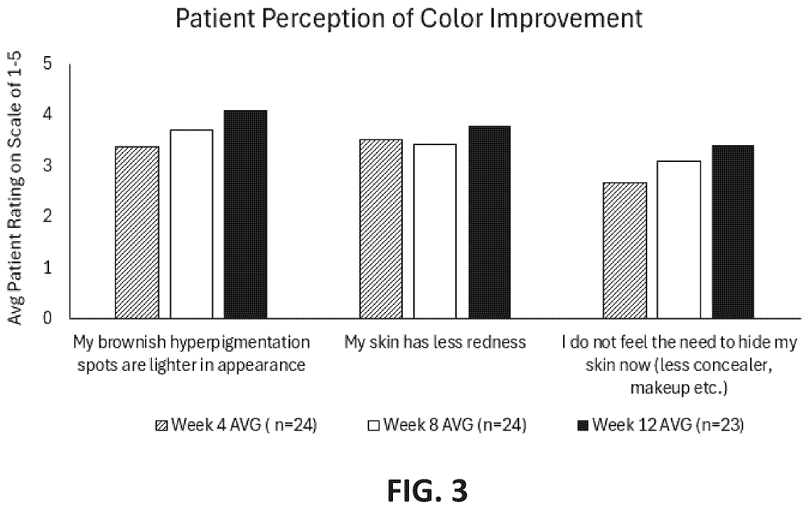

For ASG use questions, aggregate scores were not relevant; individually, (possible totals ranging between 6 and 30) for weeks 4, 8, and 12 were 34.17, 35.33, and 38.91, respectively. This denotes an average increase of 1.16 between weeks 4 and 8 (an increase of approximately 3.4%), an average increase of 3.58 between weeks 8 and 12 (an increase of approximately 10.1%), and an average increase of 4.74 between weeks 4 and 12 (an increase of approximately 12.2%). FIG. 2 provides a visual representation of these changes for three key questions addressing skin texture; FIG. 3 does the same for three key questions addressing coloration. The data for the figures was captured at weeks 4, 8, and final follow-up (week 12). Patients rated these questions on a subjective 1-5 where 1=disagree, 2=slightly disagree, 3=neither agree nor disagree, 4=slightly agree, 5=agree.

For ASG use questions, aggregate scores were not relevant. Individually, average ratings for the 6 ASG use descriptor questions throughout are shown in Table 4.

TABLE 4
Patient Perception of ASG Use

| | Average |
|---|---|
| Question | Score |
| ASG is easy to spread | 4.16 |
| Does ASG dispense easily? | 4.59 |
| ASG absorbs quickly | 3.72 |
| The scent of ASG is pleasant | 3.73 |
| ASG doesn't make my skin feel sticky | 3.64 |
| ASG doesn't make my skin feel greasy | 3.47 |

Table 4 shows data captured at weeks 4, 8, and final follow-up (week 12). Patients rated these questions on a subjective 1-5 score, where 1=disagree, 2=slightly disagree, 3=neither agree nor disagree, 4=slightly agree, 5=agree.

ASG comprising the exemplary formulation as described herein is a stable silicone gel-based topical that is easy to apply and is absorbed quickly by skin, formulated as a non-invasive therapy to improve the appearance of acne scarring and associated dark or red discoloration. Key active ingredients include 10% azelaic acid, acetyl zingerone, bakuchiol, vitamin C, licorice root extract, and medical grade silicone gel, all of which have been shown to be effective in treating acne scars and/or hyperpigmentation.

Formulations of the present disclosure allow the ingredients to remain active and stable within product formulation. The product is meant to be used twice daily, in the morning and evening.

According to the data, overall patient perception of improvements in texture and coloration as per the scoring system used was more than 10% at final follow-up (week 12) from the initial score taken at week 4. Some improvement between weeks 4 and 8 (approximately 3.4%) was noted, a more marked improvement was seen between weeks 8 and 12 (approximately 10.1%). Overall improvement was substantial (approximately 12.2%), representing a profound and promising improvement for a topical at-home therapy. Averages from individual questions followed a similar pattern for the most part.

The ASG use questionnaire elicited positive responses across all questions. In no case was the average score below 3 (neutral), indicating an overall acceptance of product qualities that suggests a high likelihood of compliance, due to the lack of qualities that may ‘turn off’ patients to regular product use. Given that the outcomes for improvement in skin coloration and texture become most readily apparent to patients after week 8 according to the data, this is essential because users must employ the formulation twice daily for a substantial length of time before results manifest. The highest rated answers were to the questions, “ASG is easy to spread” (average 4.16) and “Does ASG dispense easily?” (average 4.59) indicating the basic physical characteristics of the formulation application are its strongest attributes regarding daily use. The lowest rated average was for the question, “ASG doesn't make my skin feel greasy” (3.47), but this was still 0.47 points above neutral (score of 3 out of 5).

The results show that the formulation is safe for twice daily home use and effective for improving the appearance of acne scars, as well as reducing associated hyperpigmentation and redness. The formulation is convenient and easy to use and presents an attractive option to patients enduring acne scars, and can expect visible improvement from regular, correct use of the product.

Applicants note that the subject group included a wide range of ethnicities and included representation of all Fitzpatrick skin types. This was done to address the reality that many skin therapies will either require careful modulation of parameters or are generally avoided altogether in darker skin types (IV through VI), delineating the utility of an at-home topical skin therapy that does not require consideration of Fitzpatrick skin type when prescribed. The data was not broken down by skin type due to the sample size; such a breakdown may be revealing if a similar study were performed using a larger sample size with specific attention to this during recruitment of the subject population.

The formulation is designed to be used alone but works well adjunctively to optimize other common scar treatments such as dermabrasion, lasers, microneedling, radiofrequency, subcision or TCA cross. The formulation may also provide a ready alternative for patients who, because of skin type or any other reason, cannot or would not choose more aggressive therapies, such as lasers.

Without wishing to be bound by theory, Applicants surprising found that the formulations of the present disclosure provide a safe and effective therapeutic option for improving the appearance of acne scars and related hyperpigmentation. The formulations may be an ideal first line therapy alternative for patients with mild to moderate acne scarring regardless of skin type.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

### Embodiments

1. A composition comprising: about 0.1-2.0% by weight acetyl zingerone; about 0.1-20% by weight azelaic acid; about 0.1-3.0% by weight bakuchiol; about 0.1-10.0% by weight licorice extract; about 0.1-25% by weight vitamin C or a derivative thereof, and a silicone based additive.
2. The composition of embodiment 1, further comprising at least one of: polyethylene glycol (PEG)-30 dipolyhydroxystearate, polyglyceryl-3 diisostearate, diisopropyl adipate, ethoxydiglycol, water, ethylenediaminetetraacetic acid (EDTA), propylene glycol, glycerin, or xanthan gum.
3. The composition of embodiment 1 or 2, further comprising at least one of: petrolatum, jojoba oil, polyglyceryl-3 diisostearate, diisopropyl adipate, dimethicone, or PEG-30 dipolyhydroxystearate.
4. The composition of any one of the preceding embodiments, further comprising about 1-10% by weight niacinamide.
5. The composition of any one of the preceding embodiments, wherein the acetyl zingerone is about 0.2-1% by weight.
6. The composition of any one of the preceding embodiments, wherein the acetyl zingerone is about 0.4-1% by weight.
7. The composition of any one of the preceding embodiments, wherein the azelaic acid is about 1-15% by weight.
8. The composition of any one of the preceding embodiments, wherein the azelaic acid is about 5-12% by weight.
9. The composition of any one of the preceding embodiments, wherein the bakuchiol is about 1-2% by weight.
10. The composition of any one of the preceding embodiments, wherein the licorice extract is about 1-7% by weight.
11. The composition of any one of the preceding embodiments, wherein the licorice extract is about 2-6% by weight.
12. The composition of any one of the preceding embodiments, wherein the vitamin C or a derivative thereof is about 1-12% by weight.
13. The composition of any one of the preceding embodiments, wherein the vitamin C or a derivative thereof is about 4-12% by weight.
14. The composition of any one of the preceding embodiments, wherein the silicone based additive is about 50-70% by weight.
15. The composition of any one of the preceding embodiments, wherein the silicone based additive is about 55-65% by weight.
16. The composition of embodiment 1, comprising: about 0.2-1% by weight acetyl zingerone; about 1-15% by weight azelaic acid; about 1-2% by weight bakuchiol; about 1-7% by weight licorice extract; about 1-12% by weight vitamin C or a derivative thereof, and a silicone based additive.
17. The composition of embodiment 16, further comprising about 2-8% by weight niacinamide.
18. The composition of embodiment 1, comprising: about 0.4-1% by weight acetyl zingerone; about 5-12% by weight azelaic acid; about 1-2% by weight bakuchiol; about 2-6% by weight licorice extract; about 4-12% by weight vitamin C or a derivative thereof; and a silicone based additive.
19. The composition of embodiment 18, further comprising about 4-6% by weight niacinamide.
20. The composition of any one of the preceding embodiments, wherein the vitamin C or a derivative thereof is chosen from tetrahexyldecyl ascorbate, L-ascorbic acid, esters of ascorbic acid with fatty acids or their salts, ascorbyl palmitate, magnesium ascorbyl phosphate, sodium ascorbyl phosphate, ascorbyl glucoside, or 3-O-ethyl ascorbic acid.
21. The composition of any one of the preceding embodiments, wherein the vitamin C or a derivative thereof is tetrahexyldecyl ascorbate.
22. A composition comprising: about 0.1-2.0% by weight acetyl zingerone; about 0.1-20% by weight azelaic acid; about 0.1-3.0% by weight bakuchiol; and about 0.1-10.0% by weight licorice extract.
23. The composition of embodiment 22, further comprising at least one of: polyethylene glycol (PEG)-30 dipolyhydroxystearate, polyglyceryl-3 diisostearate, diisopropyl adipate, ethoxydiglycol, water, ethylenediaminetetraacetic acid (EDTA), propylene glycol, glycerin, or xanthan gum.
24. The composition of embodiment 22 or 23, further comprising at least one of: petrolatum, jojoba oil, polyglyceryl-3 diisostearate, diisopropyl adipate, dimethicone, or PEG-30 dipolyhydroxystearate.
25. The composition of any one of embodiments 22-24, further comprising about 1-10% by weight niacinamide.
26. The composition of any one of embodiments 22-25, wherein the acetyl zingerone is about 0.2-1% by weight.
27. The composition of any one of embodiments 22-26, wherein the acetyl zingerone is about 0.4-1% by weight.
28. The composition of any one of embodiments 22-27, wherein the azelaic acid is about 1-15% by weight.
29. The composition of any one of embodiments 22-28, wherein the azelaic acid is about 5-12% by weight.
30. The composition of any one of embodiments 22-29, wherein the bakuchiol is about 1-2% by weight.
31. The composition of any one of embodiments 22-30, wherein the licorice extract is about 1-7% by weight.
32. The composition of any one of embodiments 22-31, wherein the licorice extract is about 2-6% by weight.
33. The composition of any one of embodiments 22-32, further comprising about 0.1-25% by weight vitamin C or a derivative thereof
34. The composition of embodiment 33, wherein the vitamin C or a derivative thereof is about 1-12% by weight.
35. The composition of embodiment 33, wherein the vitamin C or a derivative thereof is about 4-12% by weight
36. The composition of any one of the preceding embodiments, for use in treating an acne scar.
37. The composition of any one of the preceding embodiments, for use in treating hyperpigmentation.
38. The composition of any one of the preceding embodiments, for use in brightening skin.
39. The composition of any one of the preceding embodiments, for use in treating wrinkles on skin.
40. A method for preparing a composition, the method comprising:
   mixing about 0.1-2.0% by weight acetyl zingerone, about 0.1-20% by weight azelaic acid, about 0.1-3.0% by weight bakuchiol, about 0.1-10.0% by weight licorice extract, and a silicone based additive to form a mixture;
   heating the mixture to 50-80 degrees Celsius;
   cooling the mixture to 40 degrees Celsius;
   combining the mixture with a ceramide complex comprising 1-5% ceramide to form a combination; and
   mixing the combination.
41. The method of embodiment 40, wherein the composition further comprises about 0.1-25% by weight vitamin C or a derivative thereof.
42. The method of embodiment 40, wherein the composition is used as an acne scar gel.
43. A method of treating a subject, the method comprising administering a composition to the subject, wherein the composition comprises about 0.1-2.0% by weight acetyl zingerone, about 0.1-20% by weight azelaic acid, about 0.1-3.0% by weight bakuchiol, about 0.1-10.0% by weight licorice extract, and a silicone based additive, wherein the method reduces melanin levels in skin of a subject relative to an untreated subject, and wherein the composition is administered as a topical formulation.
44. The method of embodiment 43, wherein the composition further comprises about 0.1-25% by weight vitamin C or a derivative thereof.
45. The method of embodiment 43, or 44, wherein the skin of the subject is lightened.
46. The method of any one of embodiments 43-45, wherein the skin of subject has a reduction of dark spots.
47. The method of any one of embodiments 43-46, wherein the skin of the subject is retextured.
48. The method of any one of embodiments 43-47, wherein the skin of the subject has improved anti-inflammatory properties.
49. The method of any one of embodiments 43-48, wherein the skin of the subject is protected against microbiomes.

Although this disclosure has been described with a certain degree of particularity, it is to be understood that the present disclosure has been made only by way of illustration and that numerous changes in the details of construction and arrangement of parts may be resorted to without departing from the spirit and the scope of the disclosure.

1. A composition comprising: about 0.1-2.0% by weight acetyl zingerone; about 0.1-20% by weight azelaic acid; about 0.1-3.0% by weight bakuchiol; about 0.1-10.0% by weight licorice extract; and a silicone based additive.

2. The composition of claim 1, further comprising at least one of: polyethylene glycol (PEG)-30 dipolyhydroxystearate, polyglyceryl-3 diisostearate, diisopropyl adipate, ethoxydiglycol, water, ethylenediaminetetraacetic acid (EDTA), propylene glycol, glycerin, or xanthan gum.

3. The composition of claim 1, further comprising at least one of: petrolatum, jojoba oil, polyglyceryl-3 diisostearate, diisopropyl adipate, dimethicone, or PEG-30 dipolyhydroxystearate.

4. The composition of claim 1, further comprising about 1-10% by weight niacinamide.

5. The composition of claim 1, wherein the acetyl zingerone is about 0.2-1% by weight.

6. The composition of claim 1, wherein the acetyl zingerone is about 0.4-1% by weight.

7. The composition of claim 1, wherein the azelaic acid is about 1-15% by weight.

8. The composition of claim 1, wherein the azelaic acid is about 5-12% by weight.

9. The composition of claim 1, wherein the bakuchiol is about 1-2% by weight.

10. The composition of claim 1, wherein the licorice extract is about 1-7% by weight.

11. The composition of claim 1, wherein the licorice extract is about 2-6% by weight.

12. The composition of claim 1, further comprising vitamin C, wherein the vitamin C or a derivative thereof is about 1-12% by weight.

13. The composition of claim 1, further comprising vitamin C, wherein the vitamin C or a derivative thereof is about 4-12% by weight.

14. The composition of claim 1, wherein the silicone based additive is about 50-70% by weight.

15. The composition of claim 1, wherein the silicone based additive is about 55-65% by weight.

16. The composition of claim 1, comprising: about 0.2-1% by weight acetyl zingerone; about 1-15% by weight azelaic acid; about 1-2% by weight bakuchiol; about 1-7% by weight licorice extract; about 1-12% by weight vitamin C or a derivative thereof, and a silicone based additive.

17. The composition of claim 16, further comprising about 2-8% by weight niacinamide.

18. The composition of claim 1, comprising: about 0.4-1% by weight acetyl zingerone; about 5-12% by weight azelaic acid; about 1-2% by weight bakuchiol; about 2-6% by weight licorice extract; about 4-12% by weight vitamin C or a derivative thereof; and a silicone based additive.

19. The composition of claim 18, further comprising about 4-6% by weight niacinamide.

20. (canceled)

21. The composition of claim 18, wherein the vitamin C or a derivative thereof is tetrahexyldecyl ascorbate.

22-39. (canceled)

40. A method for preparing a composition, the method comprising:
mixing about 0.1-2.0% by weight acetyl zingerone, about 0.1-20% by weight azelaic acid, about 0.1-3.0% by weight bakuchiol, about 0.1-10.0% by weight licorice extract, and a silicone based additive to form a mixture;
heating the mixture to 50-80 degrees Celsius;
cooling the mixture to 40 degrees Celsius;
combining the mixture with a ceramide complex comprising 1-5% ceramide to form a combination; and
mixing the combination.

41-42. (canceled)

43. A method of treating a subject, the method comprising administering a composition to the subject, wherein the composition comprises about 0.1-2.0% by weight acetyl zingerone, about 0.1-20% by weight azelaic acid, about 0.1-3.0% by weight bakuchiol, about 0.1-10.0% by weight licorice extract, and a silicone based additive, wherein the method reduces melanin levels in skin of a subject relative to an untreated subject, and wherein the composition is administered as a topical formulation.

44-49. (canceled)
